(19) United States
(12) Patent Application Publication
(10) Pub. No.: US 2008/0032915 A1
(43) Pub. Date: Feb. 7, 2008

(54) Cysteine Rich Peptides for Improving Thiol Homeostasis

(75) Inventors: Rijkje Cornelia Sprong, Ede (NL); Steffi Dudek, 's-Hertogenbosch (NL); Fiona Marie Taylor, Oneonta, NY (US)

Correspondence Address: THE WEBB LAW FIRM, P.C., 700 KOPPERS BUILDING, 436 SEVENTH AVENUE, PITTSBURGH, PA, 15219, US

(73) Assignee: CAMPINA NEDERLAND HOLDING B.V., Zaltbommel (NL)

(21) Appl. No.: 10/589,181

(22) PCT Filed: Feb. 12, 2004
(86) PCT No.: PCT/NL04/00106

§ 371 Date: Jun. 4, 2007

(51) Int. Cl. A61K 38/02; A61P 17/00; A61P 29/00; A61P 3/10; A61P 39/00; A61P 9/10; A61P 9/12

(52) U.S. Cl. 514/2

(57) ABSTRACT

Described is the use of a mixture of peptides, the peptides comprising at least 6.5% wt cysteine, for the manufacture of a medicament, supplement, beverage or food product for restoring thiol homeostasis. Furthermore, a method is described for restoring thiol homeostasis in a subject in need thereof, said method comprising administering to said subject an effective amount of a mixture of peptides, the peptides comprising at least 6.5% wt cysteine.

The present invention relates to the use of a mixture of peptides, the peptides comprising at least 6.5% wt cysteine, for the manufacture of a medicament, supplement, beverage or food product for restoring thiol homeostasis.

The physiological activity of many essential proteins including enzymes depends on the redox-state of redox-sensitive thiol groups, i.e. whether these thiol groups exist in the reduced (—SH) or oxidised (—S—S—) state. Such proteins react to very small changes in the redox potential of their environment. A whole network of non-protein biothiol antioxidants, antioxidant vitamins and antioxidant enzymes is important to maintain the structure of the proteins or to establish transfer of a redox signal to a respective target.

The so-called thiol buffer of the body comprises GSH, GSH precursors and cysteine as well as actually all redox-sensitive thiol groups in proteins. These thiols together with a number of antioxidant enzymes manage a very complex equilibrium of reduction and oxidation reactions responsible for structure and function of proteins such as enzymes. The redox chain affects for example certain signal transduction chains like the NF-kB/p50 system, receptor functions, protein kinases and phosphatases, transport function of serum albumin (such as for fatty acids), the life-time of NO by binding to thiols, and probably also some proteins regulating the apoptotic machinery.

In modern day life, almost everyone is exposed to extraneous chemical compounds on a daily base. Pollution in the atmosphere, exposure to car exhaust fumes, smoking, narcotic abuse, drinking and the intake of coffee and/or medications can all lead to a higher level of extraneous chemical compounds in he body. The intracellular thiols are molecularly and via redox status of utmost importance for the efficient removal of the negative bodily effects resulting from such higher levels of extraneous chemical compounds.

The above mechanisms involved in the thiol mediated redox status and equilibrium are encompassed in the term “thiol homeostasis”. Said term is well appreciated in the art (see e.g. D. M. Townsend, K. D. Tew and H. Tapiero, Biomed. Pharmacother. 2004, vol 58(1):47-55).

It has now surprisingly been found that a mixture of peptides, the peptides comprising at least 6.5% wt cysteine, calculated on the protein part of the peptide product, is very efficient for restoring thiol homeostasis in any subject, whether suffering from one or more pathological conditions or seemingly in pretty healthy condition. As herein used, the % wt relates to the weight of the cysteine in the total peptide weight within the mixture of peptides. The latter is conveniently calculated by a method commonly used and known in the art, namely by mulitplying the total weight percentage of nitrogen in the peptide mixture by the factor 6.38.

Thus, the present invention relates to the use of a mixture of peptides, the peptides comprising at least 6.5% wt cysteine, for the manufacture of a medicament, supplement, beverage or food product for restoring thiol homeostasis. It has been found that in subjects, in particular subjects over the age of 50, such restored thiol homeostasis conferred by the said mixture of peptides manifested itself by an improved energy and vitality, alleviation of fatigue and stress, improved sleep quality and an increased mental alertness.

As herein used, peptides are defined as amino acid chains derived from one or more proteins; the molecular weight of the peptides is preferably between 200 Da and 11,000 Da, more preferably between 300 Da and 6,000 Da, yet more preferably between 400 Da and 5,000 Da.

It is preferred that the peptides in the said mixture of peptides comprise at least 6.5% wt, more preferably at least 6.7% wt, yet more preferably at least 6.9% wt, most preferably at least 7% wt cysteine. On laboratory scale, it has been found that the cysteine content of the peptides can be as high as 20% wt; however, with large scale preparation using the current technology peptide mixtures are obtained having a cysteine content of 6.5-7.5% wt, i.e. currently the preferred range. It is however preferred that the cysteine content of the peptides is as high as possible to provide more flexibility in application and administration.

For use in a medicament or supplement, said preparation can be combined with any suitable carrier, diluent, adjuvant, excipient, etc. in order to obtain the medicament in the desired administration form. Advantageously, said medicament or supplement is administered orally. The term “supplement” is meant to include food supplements, as well as health products, such as health drinks.

For the intended use, the said mixture of peptides, the peptides comprising at least 6.5% wt cysteine, may be administered alone or in admixture with a pharmaceutically acceptable carrier.

Examples of said formulations, which may be prepared using well known methods and excipients, such as those described in “Remington's Pharmaceutical Sciences Handbook”, Mack Pub. Co., N.Y. U.S.A., are tablets, capsules, syrups, and the like for oral administration, whereas for the parenteral administration suitable forms are sterile solutions or suspensions in acceptable liquids, implants, etc.

For use in a beverage or food product, said mixture of peptides, the peptides comprising at least 6.5% wt cysteine, can be combined with any common food ingredient. The term “beverage” is meant to include cordials and syrups, as well as formulations of a dry powder to be dissolved in water or another beverage for the preparation of instant drinks.

In a preferred embodiment, said mixture of peptides, the peptides comprising at least 6.5% wt cysteine, is used for the manufacture of a supplement, beverage or food product. It is preferred that the supplement, beverage or food product is administered daily in such an amount that the dose of cysteine is 10-1000 mg, preferably of 50-600 mg, more preferably of 80-300 mg, most preferably of 100-200 mg. Through administering said mixture of peptides, it is found that said subject, either in seemingly healthy condition or suffering from a specific health condition, feels energised and in overall better shape.

In a preferred embodiment, at least 70%, preferably at least 80%, of the peptides comprises at least a terminal cysteine, which is as such readily available for the human or animal body and thus for restoring of thiol homeostasis. With this is meant that at least 70%, preferably at least 80%, of the peptides has either one or two terminal cysteines. The content of terminal cysteines can e.g. be determined by N-terminal amino acid sequencing as is known in the art.

Advantageously, the said mixture of peptides, the peptides comprising at least 6.5% wt cysteine, comprises at least 60%, preferably at least 70%, more preferably at least 80%, of the cysteines present in the cystine form. “Cystine” as herein used is the nomenclature for cysteines in oxidised form, i.e. one cysteine residue being coupled to another cysteine residue by a sulphur bridge. Throughout the present description, the term “cysteine” refers to both cysteine in the reduced form (having free SH-groups) and in the oxidised (cystine) form. Free thiols like cysteine are readily oxidised in the body leading to the generation of free radicals. Thus, high doses of free thiols could act as pro-oxidants when entering the blood stream. It is therefore preferred that the cysteine residues present in the said mixture largely exist in oxidised form, as this form is chemically less reactive, therefore safer in comparison to free cysteine when administered to subjects (See e.g. Biothiols in Health and Disease, L. Packer and E. Cadenas (eds.), Marcel Dekker Inc., New York, Basel, Hongkong (1995)).

In another embodiment, said mixture of peptides, the peptides comprising at least 6.5% wt cysteine, is used for preventing and/or reducing effects of alcohol consumption. An important intermediate in the conversion pathway of alcohol is acetaldehyde, a highly toxic compound with a high chemical reactivity towards proteins, DNA and lipids in vivo, which compound compromises the body. The formation of acetaldehyde has both long-term and short-term negative effects on the body. It is believed that thiol groups are capable of reacting directly and non-enzymatically with acetaldehyde, thus ensuring scavenging of the said toxic compound, thereby reducing the serious consequences thereof for the body. It is believed that the administration of the mixture of cysteine rich peptides results in a restoration of redox and thiol homeostasis such that thiol groups are more readily available for scavenging the toxic acetaldehyde. This is thought not only to prevent or reduce short-term effects of alcohol consumption such as a hangover and face flushing, but also long-term effects such as liver dysfunction.

Thus, in a further embodiment said mixture of peptides, the peptides comprising at least 6.5% wt cysteine, is used for preventing and/or reducing of a hangover. It is thought that reaction of thiol groups present due to restored thiol homeostasis as a consequence of ingestion of the mixture cysteine rich peptides according to the present invention with acetaldehyde results in efficient removal of the latter compound such that the consequences thereof on the body, in particular a hangover, are diminished.

In yet another embodiment, said mixture of peptides, the peptides comprising at least 6.5% wt cysteine, is used for preventing and/or reducing of face flushing. Many people, in particular those of Asian descent, possess a genetic peculiarity with respect to the activity of the enzyme acetaldehyde dehydrogenase (ALDH), which is responsible for the oxidation of acetaldehyde, the toxic metabolite of alcohol. As such, they have a deficiency in ALDH, which causes a build-up of acetaldehyde in the body causing facial flushing and other cardiovascular symptoms (H. M. Chao, Alcohol Clin. Exp. Res. 1995, vol. 19(1):104-109). Affected subjects experience face flushing even after ingesting of small amounts of alcohol, which may be experienced as quite embarrassing. It was found that ingestion of said mixture of peptides prevented and/or reduced the occurrence of face flushing and other effects caused by ALDH deficiency, such that subjects were able to feel more confident and less embarrassed in a public environment.

In again a further embodiment, said mixture of peptides, the peptides comprising at least 6.5% wt cysteine, is used for the manufacture of a medicament, supplement, beverage or food product for boosting vitality. It has surprisingly been found that subjects, even subjects not diagnosed with any health condition, felt revitalised after the ingestion of said mixture of peptides. Said subjects generally felt better and more energetic, and felt more up to life in general. It is believed that this boosted vitality and increased energy is due to counteracting the compromised thiol and redox homeostasis.

In a further embodiment, said mixture of peptides, the peptides comprising at least 6.5% wt cysteine, is used for the manufacture of a medicament, supplement, beverage or food product for preventing and/or reducing fatigue. As disclosed above, subjects generally felt more energetic and less fatigue. In an attractive embodiment, the mixture of peptides according to the present invention are also employed to reduce the symptoms of chronic fatigue.

In yet a further embodiment, said mixture of peptides, the peptides comprising at least 6.5% wt cysteine, is used for the manufacture of a medicament, supplement, beverage or food product for improving sleeping. Subjects ingesting said mixture of peptides experienced they slept better, likely due to more efficient removal of unbalancing chemical substances due to restored thiol homeostasis.

In again another embodiment, said mixture of peptides, the peptides comprising at least 6.5% wt cysteine, is used for the manufacture of a medicament, supplement, beverage or food product for preventing development of symptoms of Metabolic Syndrome, in particular for preventing development of non-insulin dependent diabetes (NIDDM). Metabolic Syndrome is believed to be caused by a combination of genetic makeup and lifestyle choices, e.g. diet and physical activity level. Usually, the Syndrome's associated medical problems develop over time. Subjects are already at risk of further developing Metabolic Syndrome when they are obese and have a high blood pressure. Metabolic Syndrome begins when insulin loses its ability to make body cells absorb glucose from the blood, such that glucose levels remain high permanently. Due to the permanently high blood glucose levels, subjects are at risk of eventually developing non-insulin dependent diabetes. The different stages of NIDDM, which are insulin resistance, hyperinsulinemia, impaired glucose tolerance, impaired fasting glucose and severe loss of β-cell function (overt NIDDM), are accompanied by manifold toxicities. It is believed that administration of the mixture of peptides according to the present invention restores thiol homeostasis and thus prevents and/or reduces the toxicities of insulin resistance in Metabolic Syndrome, in particular toxicities due to thiol mediated protein cross-linking or DNA modification (M. R. Hayden and S. C. Tyagi, J. of Pancreas 2002, vol. 3(4):86-108).

In a further embodiment, the mixture of peptides according to the present invention used to prevent and/or reduce the development of cardiovascular diseases, in particular of atheroscleropathy, i.e. the accelerated development of atherosclerosis in Metabolic Syndrome and the intermediate state on the way to overt NIDDM. It is caused by the manifold toxicities of NIDDM as disclosed above (M. R. Hayden and S. C. Tyagi, Atheroscleropathy Cardiovasc. Diabetol. 2002, vol. 1(1):3).

In another embodiment, the mixture of peptides according to the present invention is used for lowering of blood pressure. ACE converts Angiotensine I into Angiotensine II. The latter is a potent vasoconstrictor, which in case of a dysregulation leads to an increased blood pressure. It is currently believed that due to restoration of thiol homeostasis ACE activity is inhibited, such that less Angiotensine II is formed, thus ensuring lowering of blood pressure or preventing an increase in blood pressure. As such, the use of the mixture of peptides according to the present invention may also further contribute to prevent further development of Metabolic Syndrome ((R. Bataller, R. F. Schwabe, Y. H. Choi, L. Yang, Y. H. Paik, J. Lindquist, T. Qian, R. Schoonhoven, C. H. Hagedorn, J. J. Lemasters, and D. A. Brenner. J. Clin. Invest. 2003, vol. 112 (9):1383-1394).

In another embodiment, said mixture of peptides, the peptides comprising at least 6.5% wt cysteine, is used for the manufacture of a medicament, supplement, beverage or food product for preventing and/or treating drug-induced toxicity. It is believed that maintaining enzymes and low-molecular weight biothiols in the correct redox state by restoration of the thiol and redox homeostasis ensures more rapid removal of liver affecting drugs, such that the occurrence of drug-induced toxicity is avoided and/or treated.

Said mixture of peptides, the peptides comprising at least 6.5% wt cysteine, can also be used for the manufacture of a medicament, supplement, beverage or food product for lightening of skin. The major factor determining skin colour is the concentration and admixture of types of melanines, i.e. eumelanin (black/brown pigments) and pheomelanin (amber/red pigments), in the melanocytes of a subject. It is currently believed that said mixture of peptides modulates the activity of tyrosinase, one of the early enzymes in the pathway to melanins, such that less melanins are produced, resulting in the lightening of skin. Moreover, it is believed that intracellular thiol groups are able to modulate the proportion of eumelanin and pheomelanin in favour of pheomelanine resulting in a lighter pigment. Since said mixture of peptides also increases the concentration of thiols within the cells, it may shift the synthesis of melanins in the direction of the lighter pigment pheomelanine. Visuable effects are particularly expected in skin types with an approximately 1:1 ratio between both pigments. The mixture of peptides according to the present invention may also be used for the oral treatment of local skin discoloration as it may occur during scar formation in darker skin types after inflammatory conditions such as acne (R. M. Halder, H. L. Brooks, and V. D. Callender, Dermatol. Clin. 2003, vol. 21 (4) :609-615).

In yet another embodiment, said mixture of peptides, the peptides comprising at least 6.5% wt cysteine, is used for reducing inflammation. The inflammatory process itself is accompanied with a higher demand for sulphur containing amino acids for the maintenance of the acute phase protein synthesis and the immune cell activity, to counteract the generation of free radicals due to inflammation, and later on for the restoration of the damaged tissue. Chronic inflammation may lead to a consistent dysregulation of thiol and redox homeostasis resulting in severe systemic consequences. It is believed that the mixture of peptides according to the present invention shows beneficial effects in inflammation conditions like arthritis, chronic inflammatory bowel syndrome, acne and sepsis (F. Santangelo, Curr. Med. Chem. 2003, vol. 10(23):2599-2610).

In a preferred embodiment, said mixture of peptides, the peptides comprising at least 6.5% wt cysteine, is obtained by a method comprising the steps of:

a) cleaving proteins of a protein source into peptides;

b) digesting the peptides obtained in step a) by at least one exopeptidase, the action of which is at least attenuated at the position of a cysteine in the peptide, therewith forming digested peptides having a terminal cysteine;

c) purifying the digested peptides.

In the first step a) proteins of the protein source are cleaved into smaller peptides. This cleavage can be performed by cleavage reactions known in the art; preferably, the cleavage is performed by enzymatic hydrolysis of the peptide bonds of the protein by e.g. an endopeptidase, resulting in the peptides of about the desired length, and therewith increasing the amount of substrate for the exopeptidase. An example of an endopeptidase suitable for the present purpose is Alcalase from NOVO Nordisk.

In the second step, the peptides as obtained by the cleavage reaction are digested by at least one exopeptidase. With “at least one exopeptidase” is meant that the digestion reaction can be carried out by one or more different exopeptidases. Exopeptidases release single amino acids from the terminal ends of the peptides. The exopeptidase and the digestion reaction conditions are chosen such that the exopeptidase action is at least attenuated at the position of a cysteine in the peptide. With “at least attenuated” is meant that the exopeptidase does not remove the cysteine from the peptide at the chosen reaction conditions or has very low preference for the cleavage of cysteine, therewith rendering said cleavage reaction very slow compared to cleavage of other amino acids from the peptide. By the use of such an exopeptidase and conditions, peptides are generated from which the terminal amino acids have been removed up to the cysteine residue most close to said terminus. The skilled person will be able to find conditions at which commercially available enzymes with exopeptidase function have attenuated action at the cysteine. It is to be understood that the peptides may have one or more amino acid chains that are coupled to each other by disulfide bridges of cysteine residues, present in the said amino acid chains. “A digested peptide having a terminal cysteine” therefore reflects to the fact that at least one of the termini of such a multi-chain peptide has a terminal cysteine. Of course, such a peptide may contain more than one terminal cysteine. Preferably, the enzymatic activity is inactivated before the purification step, e.g. by a pH shift or a thermal heat inactivation treatment.

Preferably, the exopeptidase comprises Carboxypeptidase Y (E.C. 3.4.16.1.), as it has been found that this enzyme can be very effectively attenuated at cysteine residues, therewith producing peptides with terminal cysteine residues.

The cleavage step a) and the digestion step b) can be conducted simultaneously, e.g. by using an endopeptidase and an exopeptidase that both function at the same reaction conditions. Also, enzyme preparations can be used that have both endopeptidase and exopeptidase activity.

Finally, these digested peptides are purified. Suitable methods to separate the digested peptides from free amino acids released by the exopeptidase are known in the art. Since a difference in molecular weight is created between the cysteine containing peptides and the free amino acids released by the action of the exopeptidase, the cysteine rich peptides can be purified using this difference. Several techniques known in the art could be used for this purpose. Preferably, the free amino acids and other low molecular weight compounds are removed using a membrane process, preferably ultrafiltration, diafiltration or nanofiltration. The purification step can also advantageously comprise the use of an immobilised metal affinity chromatography step (IMAC) accordingly to Kronina et al., Journal of Chromatography A, 852 (1999) pp 261-272. The cysteine rich peptides can hereafter be dried.

In a special embodiment, the exopeptidase in step b) and the cleavage reaction are chosen such, that the exopeptidase is at least attenuated at the position of a cysteine in the peptide. This will result in digested peptides having predominantly a terminal cysteine.

The protein source may be any source as long as it comprises cysteine-containing proteins. The protein source can also be prepared before being subjected to the method of the present invention, by e.g. two or more protein sources before or during the cleavage step.

Preferably, the protein source consists of edible proteins, so that the digested peptides can be used as food additive. In a very special embodiment, the protein source comprises whey protein isolates (WPI) and/or whey protein concentrates (WPC). The terms “whey protein isolates” and “whey protein concentrates” are known in the field. Whey protein concentrate is a whey protein product having 35-80 w/w % protein, whereas whey protein isolate has a protein content of 90 w/w % or higher. An example of WPC 80 is Alacen 132 from Tatua (New Zealand); an example of WPI is Bipro from Davisco Foods International (USA), or Acid Whey Protein Isolate from ARLA Foods, Denmark. Whey protein isolate comprises very suitable cysteine rich proteins, such as albumin, especially (α-lactalbumin, and bovine serum albumin. Said proteins are advantageously used in or as starting protein source of the method according to the invention.

In another preferred embodiment, the protein source comprises one or more of the group consisting of albumin, especially α-lactalbumin, bovine serum albumin, egg proteins (e.g. ovalbumin, cystatin), wheat gluten, maize protein isolate, γ-conglutin (lupin), and rapeseed albumin.

Preferably, steps a) and b) are performed under conditions allowing sulphur bridges between cysteine residues as present in the proteins in the protein source to be kept in the oxidised form as much as possible. In this way, cysteine rich peptide mixtures are obtained, in which most of the cysteine residues are oxidised and coupled to other peptides through disulphide bridges. The oxidised form of said peptide mixtures is less reactive and therefore more stable in applications. A further advantage is the fact that many enzymes having exopeptidase activity do not cleave oxidised cysteines, whereas cysteines in reduced form may be cleaved from the peptides by said enzymes, albeit with a relative low activity. In order to avoid side chain modifications in the peptide mixtures, steps a) and b) are preferably conducted at a pH between 2 and 8.

It is preferred to carry out the hydrolytic processes in acidic environments. At acid pH the disulphide bridges in cystine are more stable than at basic pH. [Creighton, T. E., 1993, Proteins: structures and Molecular Properties. 2^{nd} Ed.; Freeman and Company, New York]

In a very attractive embodiment, the enzyme with endopeptidase function also has exopeptidase function, the exopeptidase function of which is attenuated at the position of cysteine. Such enzymes are known in the art and the advantage thereof is that steps a) and b) can be done simultaneously. Examples of preferred enzymes having both endopeptidase as exopeptidase functions are Flavourzyme (NOVO Nordisk), Acid Protease A, Protease M, Protease 2A, Protease B (Amano Enzyme), Corolase PN-L (AB Enzymes, UK), Enzeco Acid Fungal Protease (EDC, USA) or a combination of two or more thereof.

Preferably, at least 70%, more preferably at least 80%, of the peptides of the preparation comprises terminal cysteines, which are then readily available for the human or animal body. These terminal cysteines are obtained by the use of the exopeptidase as discussed above.

In a further aspect, the present invention relates to a method for restoring thiol homeostasis in a subject in need thereof, said method comprising administering to said subject an effective amount of a mixture of peptides, the peptides comprising at least 6.5% wt cysteine. Said method is advantageous for reasons that are disclosed above.

In a preferred embodiment, said method is for preventing and/or reducing effects of alcohol consumption in a subject in need thereof, in particular for preventing and/or reducing of a hangover and for preventing and/or reducing of face flushing. Said method is advantageous for reasons that are set out above.

In another embodiment, said method is for boosting vitality, in particular for preventing and/or reducing fatigue, in particular symptoms of chronic fatigue, for reasons outlined above.

Said method can also advantageously be employed for improving sleeping. It was found that subjects having been administered an effective amount of a mixture of peptides, the peptides comprising at least 6.5% wt cysteine, were capable of sleeping better, as is also discussed above.

Alternatively, said method can be applied for preventing development of symptoms of Metabolic Syndrome, in particular of non-insulin dependent diabetes (NIDDM) as well as for preventing and/or reducing the development of cardiovascular diseases, in particular of atheroscleropathy, for reasons as indicated above.

In yet another embodiment, said method may be employed for lowering of blood pressure. Said method is advantageous for reasons that are outlined above.

Said method can also be used for preventing and/or treating drug-induced toxicity as has been discussed above.

In a further embodiment, said method may be employed for lightening of skin, as discussed above.

In again a further embodiment, said method may be used for reducing inflammation, such as in acne.

It is preferred that in the above method said mixture of peptides, the peptides comprising at least 6.5% wt cysteine, is obtained by a method comprising the steps of:

a) cleaving the proteins of a protein source into peptides;

b) digesting the peptides obtained in step a) by an exopeptidase, the action of which is at least attenuated at the position of a cysteine in the peptide, therewith forming digested peptides having a terminal cysteine;

c) purifying the digested peptides.

A highly advantageous mixture of peptides is obtained having a large proportion of terminal cysteine residues which are readily biologically available as to restore thiol homeostasis. Said method can be conducted using any of the embodiments disclosed above.

The present invention will now be illustrated in more detail by means of the following non-limiting examples. The percentages indicated therein are weight percentages, unless indicated otherwise.

### EXAMPLES

### Example 1

### Process For The Preparation Of Cysteine Rich Peptides

A 5% wt dispersion of whey protein isolate (WPI; typically Bipro, Davisco) was produced by adding the WPI into pre-heated demineralised water followed by heating to process temperature (50° C.). The pH was adjusted to pH 3 by adding 30% sulphuric acid. The hydrolysis was initiated by adding ENZECO fungal acid protease (EDC, U.S.). The enzyme/protein ratio was typically 2% wt, based on protein dry matter. After an appropriate hydrolysis time, typically 20 h, NaOH (33%) was added until the pH reached 6.5, followed by heating the mixture to 104° C. and holding the temperature for 3 min. The hydrolysate was subjected to diafiltration (typically 300%, optionally 200%) with demineralised water at 50° C., following volume reduction and nanofiltration, using a Nadir SS NF-PES-10 3838 B membrane module. The nanofiltration proceeded at 50° C. When a dry matter of typically 25% was reached, the retentate was spray-dried.

### Example 2

### Typical Analysis

The analysis was performed by a certified commercial laboratory (CCL Nutricontrol, Veghel, The Netherlands). Method: oxidation of the cysteine in the sample with performic acid prior to HCL hydrolysis of the peptides, ion-exchange chromatography of the free amino acids following post-column derivatisation with ninhydrine, according to EC Guideline 98/64/EG of Mar. 9, 1998; Publication L257/14-23 dated 19 Sep. 1998)

| | | typical |
|---|---|---|
| Total solids | % | 95.1 |
| Protein (N*6.38) | % | 83.4 |
| Protein (N*6.62) | % | 86.0 |
| Cysteine | g/kg | 65.50 |
| Cysteine | %/Prot | 7.9% |
| Amino acids | | |
| Alanine | g/kg | 37.0 |
| Arginine | g/kg | 18.6 |
| Aspartic acid | g/kg | 146.9 |
| Glutamic acid | g/kg | 186.4 |
| Glycine | g/kg | 17.7 |
| Histidine | g/kg | 19.6 |
| Isoleucine | g/kg | 43.0 |
| Leucine | g/kg | 59.4 |
| Lysine | g/kg | 89.8 |
| Methionine | g/kg | 12.2 |
| Phenylalanine | g/kg | 18.4 |
| Proline | g/kg | 37.6 |
| Serine | g/kg | 43.3 |
| Threonine | g/kg | 47.3 |
| Tryptophan (after enzymatic hydrolysis) | g/kg | 11.4 |
| Tyrosine | g/kg | 18.6 |
| Valine | g/kg | 45.6 |
| Total a.a. | | 915.29 |

Free thiol groups in the peptides were determined with DTNB (Ellmann's reagent) in the presence of urea, with reduction by NaBH₄. The ratio of SH/SS was calculated by the concentration of available thiols without reduction/total concentration of thiols after reduction and was found to be 6.5%

### Example 3

### Molecular Weight Distribution

### Method:

### HPLC-system: isocratic HPLC system with UV detector, autosampler, Waters Millenium Data Acquisition Software

column: Progel TSK-G2000SWXL 7.8 mm×30 cm (Supelco), guard column: Progel TSK SWXL (Supelco)
eluent: 30% actetonitrile/H₂O/0,1% TFA

### Flow: 1 mL/min

### Detection: 214 nm

### Calibration: HPLC standards (Sigma): Carbonic Anhydrase, Ribonuclease A, Aprotinin, Insulin, Bacitracin, Phenylalanine,

The molecular weight distribution was found to be as follows:

| MW-range (D) | Area % |
|---|---|
| >10.000 | 17.5 |
| 10.000-5.000 | 3.7 |
| 5.000-2.000 | 6.6 |
| 2.000-1.000 | 12.1 |
| 1.000-500 | 20.2 |
| <500 | 39.9 |

### Example 4

### Preparation of Tablets Comprising The Cysteine Rich Peptides

### Dosage: 4 tablets/day, delivering 200 mg L-cysteine/day in the form of a peptide mixture.

| | Per 100 g |
|---|---|
| Cysteine rich peptides | 88.24 g |
| Microcristalline cellulose ¹ | 10.59 g |
| Silicon Dioxide ² | 0.47 g |
| Magnesium Stearate | 0.35 g |
| Stearic Acid | 0.35 g |

¹ Avicel PH-102 - FMC
² CAB-O-SIL M-5

The powder were premixed whilst the Mg stearate was withheld for the last minutes of mixing. The tablets were prepared by direct compression (compression pressure 20 kN, hardness: 160 N).

### Example 5

### Preparation Of A Chocolate Caramel Bar Comprising Cysteine Rich Peptides

### One bar delivers 200 mg L-cysteine in the form of a cysteine rich peptide mixture.

| | Per 100 g | | per Serving (40 g) | |
|---|---|---|---|---|
| Cysteine rich peptides | 8.30 | g | 3.32 | g |
| Maltitol Syrup ¹ | 66.01 | g | 26.41 | g |
| Calcium Caseinate | 10.65 | g | 4.26 | g |
| Chocolate Liquor | 8.52 | g | 3.41 | g |
| Sodium Caseinate, granular | 4.26 | g | 1.70 | g |
| Cocoa Butter | 2.13 | g | 0.85 | g |
| Butter, Unsalted | 0.11 | g | 0.04 | g |
| Lecithin | 0.01 | g | 0.0043 | g |
| Vanilla Flavor | 0.01 | g | 0.0043 | g |

¹ Lycasin 85% solution

### Example 6

### Preparation Of A Heat-Treated Yoghurt Drink Comprising Cysteine Rich Peptides

| | Per 100 g | | L-cysteine/serving |
|---|---|---|---|
| Cysteine peptide | 0.86 | g | 0.05 g |
| Skimmed milk | 55 | g | |
| Sugar | 6 | g | |
| Maltitol ¹ | 3 | g | |
| Lactic acid | 0.002 | g | |
| Pectin ² | 0.3 | g | |
| Flavor | 0.055 | g | |
| Water | 34.6 | g | |
| Inoculum ³ | 0.2 | g | |

¹ C*maltidex 16385 Cerestar
² Genu Pectine YM-115-H CP Kelco
³ YC-X11 Christian Hansen

Milk was mixed with water. The cysteine rich peptides, sugar and maltitol were added and dissolved with continuous stirring followed by pasteurisation (90° C., 5 min). After cooling to the fermentation temperature (42° C.), the inoculum was added. Fermentation was proceeded until the pH reached 4.3. The pH was lowered to 3.8-4.0 using lactic acid. The pectin was added under vigorous stirring. The mixture was heated to 70° C., homogenised at 120/20 bar and flavour was added. After filling, the product was pasteurised (80° C./3 min).

### Example 7

### Preparation Of A Liver Cleansing Drink Comprising Cysteine Rich Peptides

| | | | per Serving | | |
|---|---|---|---|---|---|
| | Per 100 g | | (250 ml) | | L-cysteine/serving |
| Cysteine peptide | 0.43 | g | 1.08 | | 0.06 g |
| Dextrose | 4.4 | g | 11 | g | |
| Fructose | 3.6 | g | 9 | g | |
| Flavor apple | 0.055 | g | 0.14 | | |
| Flavor banana | 0.037 | g | 0.09 | | |
| Pectin ¹ | 0.15 | g | 0.37 | | |
| Water | 91.33 | g | 228.36 | | |

¹ Genu Pectine YM-115-H CP Kelco

All dry ingredients were dissolved in water and the pH was adjusted first till pH 3,8 with citric acid (+/−0,14% on total), than till pH 3,5 with malic acid (+/−0,66% on total). The solution was preheated to 70° C. followed by addition of the pectin premix (4% in water). After homogenisation (150 bar) the product was filled and pasteurised or pasteurised and filled aseptically.

### Example 8

### Reduced Hepatotoxicity Of Paracetamol After Administration Of Cysteine Rich Peptides

Rats were fed an isocaloric and isonitrogenic diet containing the maintenance concentration sulphur containing amino acids (38 mg/kg) or the respective diet enriched with cysteine rich peptides (62 mg/kg) for 14 days. After 14 days, the animals in each diet group were challenged with acetaminophen (orally, 300 mg/kg body weight, in corn oil) and food was deprived for the following 12 h. Immediately after the challenge as well as 12 and 24 hours, 6 (t=0) and 9 rats (t=12 h and t=24) of each group were sacrificed and the livers withdrawn for biochemical analysis and histological examination. Blood samples were taken for analysis of plasma liver marker enzymes. Paracetamol led to a liver tissue damage both in the control group and the group receiving cysteine rich peptides, but continued supply of cysteine rich peptides improved the capacity of the rat liver to restore its structural integrity significantly as compared with the control group.

### Liver aspartate aminotransferase (ASAT)[U/L].

The specific activity of the liver enzyme aspartate aminotransferase is a relevant indicator for liver damage. If the level of activity of the enzyme is increased, this indicates liver damage.

| Hours after | | Cysteine rich peptides |
|---|---|---|
| challenging | Casein diet | enriched diet |
| 0 | 69 +/− 2 | 78 +/− 6 |
| 12 | 126 +/− 14 | 93 +/− 15 |
| 24 | 120 +/− 16 | 109 +/− 16 |

### The table above clearly indicates that liver damage by paracetamol is substantially reduced by taking a diet enriched in cysteine rich peptides.

### Necrotic cells [cells/20 fields] Microscopic histological examination

| | | | | Cysteine rich peptides | | |
|---|---|---|---|---|---|---|
| Hours after | Casein diet | | | enriched diet | | |
| challenging | Median | Min | Max | Median | Min | max |
| 0 | 2.5 | 0 | 4 | 4 | 1 | 14 |
| 12 | 51 | 2 | 229 | 61 | 1 | 376 |
| 24 | 16 | 0 | 89 | 3 | 1 | 9 |

From the table shown above it can be observed that the number of necrotic cells, after an initial rise, eventually comes down in the cysteine rich peptides enriched diet group to much lower levels compared to the control (casein) group, indicating a quicker restoration of the tissue integrity, as a result of restored biothiol homeostasis.

### Vacuolated cells [cells/20 fields] Microscopic histological examination.

| | | | | Cysteine rich peptides | | |
|---|---|---|---|---|---|---|
| Hours after | Casein diet | | | enriched diet | | |
| challenging | Median | Min | Max | Median | Min | max |
| 0 | 16 | 1 | 146 | 4 | 0 | 13 |
| 12 | 80 | 16 | 824 | 419 | 10 | 618 |
| 24 | 11 | 2 | 141 | 7 | 0 | 32 |

Vacuolated cells signify the beginning of cell damage, eventually leading to cell necrosis. Vacuolation of cells is a reversible process. From the table above it can be concluded that a cysteine rich peptides enriched diet leads to a quicker reversal to normal state.

### Example 9

### ACE Inhibition Activity Of Cysteine Rich Peptides

The ACE inhibition assay is based on the hydrolysis of furylacryloyl-phenylalanyl-glycyl-glycine (FAPGG) as a substrate according to Maguire and Price (Ann. Clin. Biochem. 1985, vol. 22: 204-210), adapted for a microtiter procedure. ACE from rabbit lung (nr. A6778), FAPGG (nr F7131) and Captopril® (nr. C 8856) were obtained from Sigma. Different concentrations of the test substances (inhibitor) were added to the substrate solution (0.145 mmol), the reaction was started by adding the enzyme (ACE, 0.145 U). The decrease in absorbance at 340 nm was measured during 10 minutes at 1 minute intervals with a μQuant plate reader from Bio-Tek instruments. The IC50 was obtained from the plot of the inhibitor concentration vs. ACE inhibition (%). Whey protein isolate (Bipro, Davisco) was taken as a negative control. The IC 50 is defined as the inhibitor concentration causing 50% inhibition of ACE.

### Results:

IC 50

Cysteine rich peptides:

| 300% diafiltration of the hydrolysate (ex. 1) | 203.23 | mg/L |
|---|---|---|
| 200% diafiltration of the hydrolysate (ex. 1) | 133.47 | mg/L |
| Captopril ® | <2 | mg/L |
| Whey protein isolate (Bipro, Davisco) | no inhibition | |
| CE 90 B | 80 | mg/L |

Captopril® is a well known ACE inhibitor. Reference CE 90 B is a casein hydrolysate from DMV International (The Netherlands) showing ACE inhibitory activity.

### Example 10

### Human Study Of Effects Of Cysteine Rich Peptides

13 people aged 50+ were asked to take cysteine peptide pills for 4 weeks in a dose of 3.3 g of the micture of cysteine rich peptides corresponding to 200 mg cysteine/day). Before and after the test, the health concerns and status were studied by means of questionnaires and interviews.
9 people finished the study. 7 of them observed positive effects which were described as follows:
increased natural level of energy
better sleep quality (more refreshed in the morning)
improved metal alertness, better attention
three people in the study with elevated blood pressure reported a decrease, which is consistent with the results of example 8.

### The effects of increased energy were documented 4-5 days after the start of the intake, and energy levels were reported to have declined after having stopped taking the pills.

10 people of different age (30-50) but exposed to a high stress level took the same dose of the mixture of cysteine rich peptides but without the thorough examination.
The observations reported by the test persons were: better sleep (more refreshed in the morning), and more energy during the day.
5 adult people of Asian origin, which all described themselves as being hangover-sensitive, took 0.8-1.6 g of the mixture of cysteine rich peptides (comprising 6.5% wt cysteine) directly preceding alcohol consumption. All subjects reported that they neither suffered from a hangover, nor were they experiencing any embarrassing face-flushing.
2 adult people of Asian origin took 0.8-1.6 g of the mixture of cysteine rich peptides (comprising 6.5% wt cysteine) during at least 4 weeks. The subjects reported that their skin condition and skin pattern had improved (reduced acne). Moreover they reported to have more energy, and slept better.

1-28. (canceled)

29: A method for restoring thiol homeostasis in a subject in need thereof, comprising administering to said subject an effective amount of a mixture of peptides comprising at least 6.5% wt cysteine.

30: The method according to claim 29, wherein said mixture of peptides is administered to prevent and/or reduce effects of alcohol consumption in a subject in need thereof.

31: The method according to claim 30, wherein said mixture of peptides is administered to prevent and/or reduce a hangover.

32: The method according to claim 30, wherein said mixture of peptides is administered to prevent and/or reduce face flushing.

33: The method according to claim 29, wherein said mixture of peptides is administered to boost vitality.

34: The method according to claim 33, wherein said mixture of peptides is administered to prevent and/or reduce fatigue.

35: The method according to claim 29, wherein said mixture of peptides is administered to improve sleeping.

36: The method according to claim 29, wherein said mixture of peptides is administered to prevent development of symptoms of Metabolic Syndrome such as non-insulin dependent diabetes.

37: The method according to claim 36, wherein said mixture of peptides is administered to prevent and/or reduce the development of cardiovascular diseases such as atheroscleropathy.

38: The method according to claim 29, wherein said mixture of peptides is administered to lower blood pressure.

39: The method according to claim 29, wherein said mixture of peptides is administered to prevent and/or treat drug-induced toxicity.

40: The method according to claim 29, wherein said mixture of peptides is administered to lighten skin.

41: The method according to claim 29, wherein said mixture of peptides is administered to reduce inflammation.

42: The method according to claim 29, wherein said mixture of peptides comprising at least 6.5% wt cysteine is obtained by a method comprising the steps of:
a) cleaving proteins of a protein source into peptides;
b) digesting the peptides obtained in step a) by an exopeptidase, the action of which is at least attenuated at a position of a cysteine in the peptide, therewith forming digested peptides having a terminal cysteine; and
c) purifying the digested peptides.
